(19) United States
(12) Patent Application Publication
(10) Pub. No.: US 2010/0202987 A1
(43) Pub. Date: Aug. 12, 2010

(54) PREPARATION FOR IMPROVING THE PROTECTION OF HUMAN CELLS, ESPECIALLY CELLS OF THE HUMAN SKIN, FROM THE HARMFUL INFLUENCES OF OXIDATIVE NOXAE AND UV RADIATION

(76) Inventors: Jean Krutmann, Wegberg (DE); Judith Haendeler, Gladbach (DE)

Correspondence Address: DON J. PELTO;Sheppard, Mullin, Richter & Hampton LLP, 1300 I STREET, NW, 11TH FLOOR EAST, WASHINGTON, DC, 20005, US

(21) Appl. No.: 12/668,675

(22) PCT Filed: May 1, 2008
(86) PCT No.: PCT/US08/05587

§ 371 Date: Apr. 14, 2010

(30) Foreign Application Priority Data
May 18, 2007 (US) 11/804506

(51) Int. Cl. A61K 8/72

(52) U.S. Cl. 424/59

(57) ABSTRACT

The invention relates to topical preparations for improving the protection of the mitochondria of human skin cells from the deleterious influences of oxidative noxae and UV irradiation, said preparations being characterised by a content in proteins that are capable of permeating the mitochondrial membrane. The invention also relates to a corresponding cosmetic method for treating human skin.

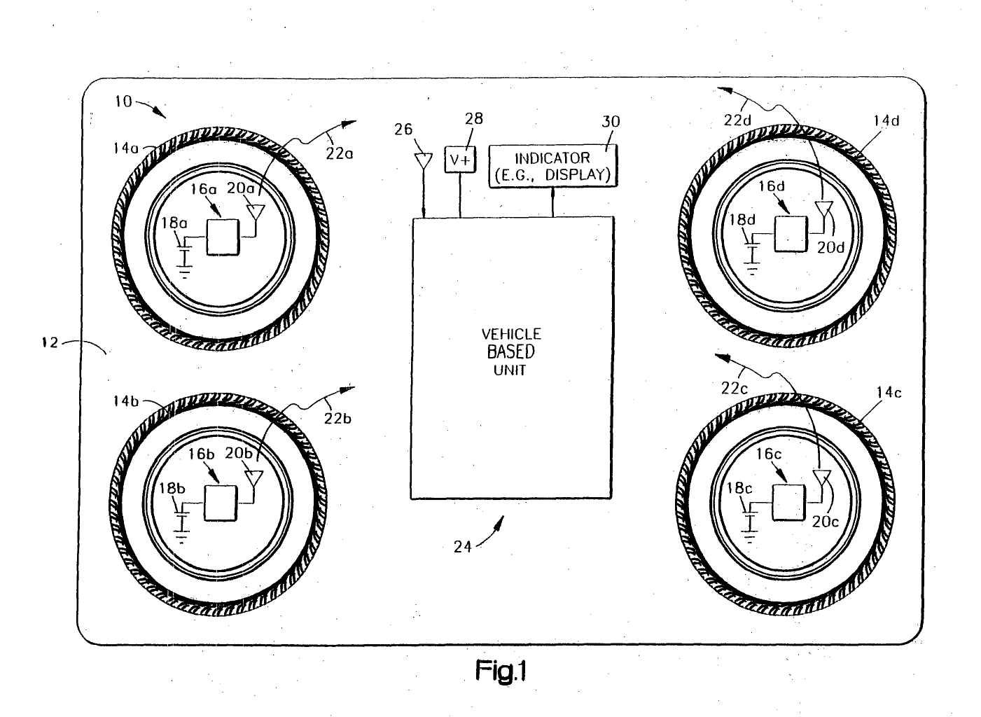

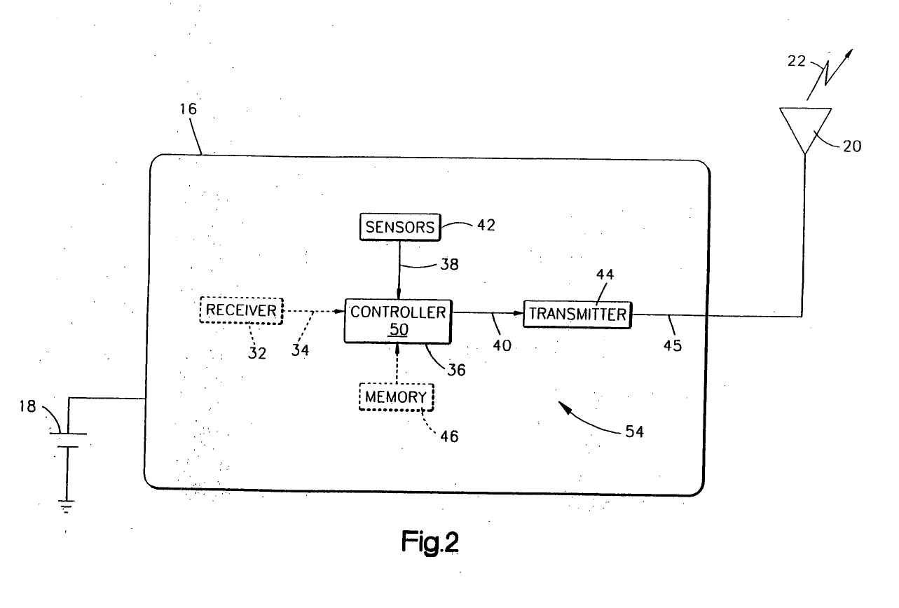

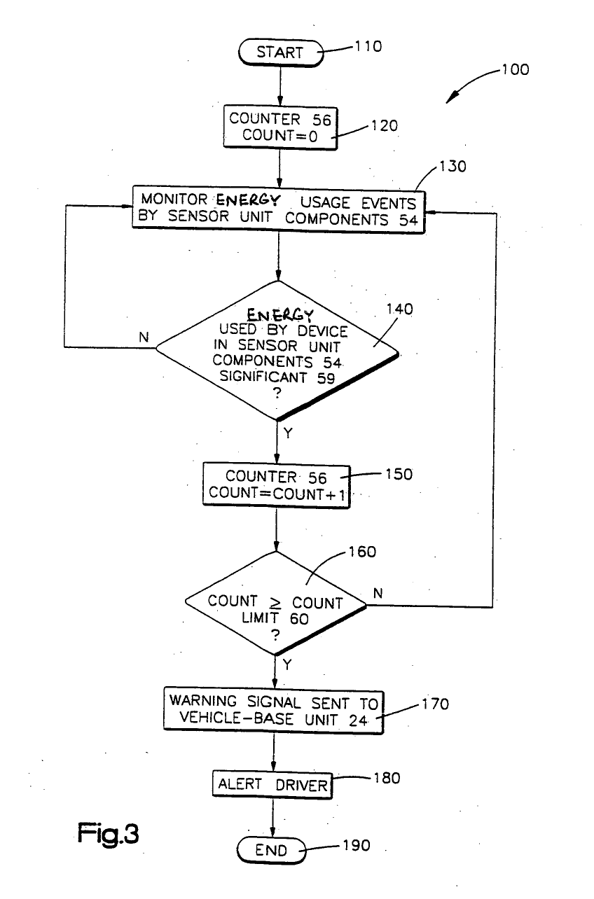

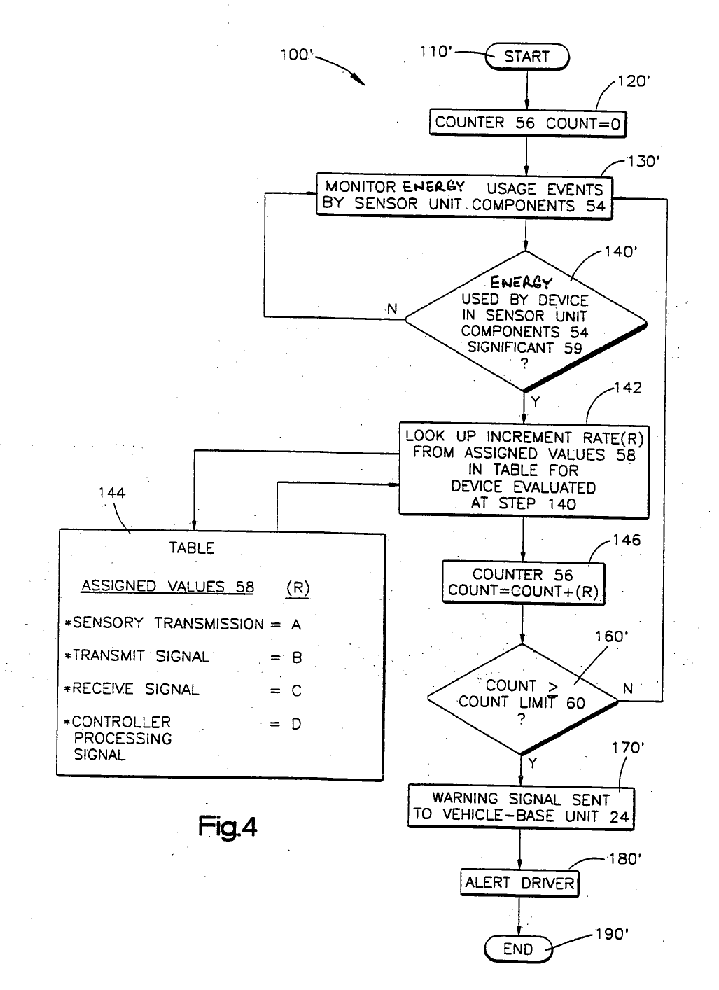

The invention relates to preparations for the improvement of the protection of mitochondria of human cells, in particular of mitochondria of cells of the human skin, from deleterious influences caused by oxidative noxae and ultraviolet irradiation.

Mitochondria are intra-cellular organelle which are extraordinarily important elements for the production of energy of the cell. The mitochondria ensure the degradation of the fatty acid, and the citric acid cycle, the electron transport chain and the oxidative phosphorylation take place here. For these processes, the organelle are provided with a complete set of enzymes. So to speak, mitochondria are the power plants of the human cells, and thus are responsible for the entire energy conversion of the cell metabolism. On account of their internal structure, mitochondria are extraordinarily sensitive to toxic environmental influences and to the irradiation of ultraviolet light of certain wave lengths (UVA and UVB).

Under the influence of these noxa and stress factors, the presence of oxygen causes radicals and so-called reactive oxygen species (ROS), which impair the ability of the mitochondria to convert energy correctly. These impairments and other factors, such as cytochrome C, are responsible for the fact that the efficiency of the mitochondria continuously decrease with the age of the cells, and in the end lead to cell death in the form of apoptosis under the influence of cytochrome C. These processes are perceived as natural aging.

The weakening of the mitochondria now results in a further increase of the ROS so that in the end they cannot use the natural protection mechanism of the corresponding enzymes and further accelerate the cell death. During the course of the oxidative processes mentioned above, radicals act accelerating, in particular those which are formed from hydrogen peroxide compounds.

The influence of free radicals on the aging process of human cells is considered well established in the meantime. From a schematic point of view, in the process described above free radicals are produced primarily as a waste product of endogenic energy production during the oxidation from hydrogen to water. The said occurs within the cause of the electron transport chain, when the electrons escape from the transport chain and are entrapped by oxygen atoms or if the oxidation is not carried out completely and the inert-gas shell of the oxygen atoms aimed at is achieved partially only during the junction with hydrogen. The developing product in the form of a peroxide or hydroxyl radical is extraordinarily reactive now and seeks random atoms as reactants, provided they can contribute to an energy saturation of the inert-gas shell. In the process, cytopathogenic by-products occur. In the body, they initiate a kind of chain reaction, in which the “fragments” try to acquire the electron missing for the formation of the complete outer shell of the oxygen atom by connecting up to the neighbouring molecules, such as lipids or DNA, and thus changing them.

The most sensitive weak point for radicals are mitochondria. The destructive action develops there because the mitochondrial membrane as well as the mitochondrial DNA (mt-DNA) is damaged in particular. It reacts especially sensitively to oxidative attacks. As it is coded for well over a dozen proteins, which are of significance for the function of energy production, the damage is considerable. Whilst the chromosomal core DNA has a number of enzyme systems which are able to cut out oxidised DNA fragments and to replace them, the mt-DNA, which is older looking at it from a biological evolutionary point of view, does not have such corrective systems. In addition, it also lacks histones, which can screen genetic material otherwise. For this reason, the oxidative damage is especially dramatic on the mitochondria. A vicious circle occurs because the damaged mitochondria produce less and less ATP, the energy carrier of all cell biological process, and at the same time they increasingly release free radicals as waste products which in turn now cause further damage to the mitochondria.

Thus, the cell has less and less energy available. The damage induced by the free radicals accumulates, which results in an increasing loss of function of the mitochondria so that the aging of tissue and organs, in particular of the largest organ of humans, the skin, becomes visible from the outside as well.

For more than fifty years, free radicals have been considered the key factors of the aging process, and many times before attempts have been made to prevent the well-established influence on the aging process as far as possible. In keeping with “mitochondrial medicine”, there is an approach of anti-aging medicine which is geared to the protection and/or repair of these cell organelle. At present, however, this approach is in an especially difficult process as researchers have not managed yet to completely explain the enzyme processes involved in the repair of damaged mitochondrial DNA.

Although EP-A 1 382 327 revealed that the human skin can be protected against aging and against deleterious influences caused by environmental toxins and UV irradiation by producing and applying cosmetic or pharmaceutical preparations which increase the synthesis of the energy donators of the mitochondrial electron transport chain and at the same time decrease the rate of reactive oxygen species (ROS) in cellular metabolism. In this way, the human skin cells are to be stimulated as a protection against the noxae mentioned.

The theory derived from EP-A 1 382 327 says that the activation of mitochondria by energy donators of the human respiratory chain (such as ATP, creatin phosphate, glucose-6-phosphate, pyrophosphate and phosphoenolpyruvate), i.e. compounds, which due to their special structure take over the transfer of chemically bonded energy between energy-supplying and energy-consuming processes without causing an increase of ROS at the same time, can be increased by using vegetable extracts and different active substances, preferably antioxidants, such as a combination of ascorbic acid, yeast and glycogen and at least one other active compound. The mixture mentioned last is to exert a synergistic effect on the energy balance of the cell, thus protecting it against the damage mentioned [0015-0017].

Amongst the many active antioxidants [0018], structurally completely different substances are mentioned, but there are no proteins amongst them. Up to now, attention seems to have been given primarily to a direct influence on the energy balance of the cell. In this respect, the fact has been ignored that in the proposed procedure all ROS without any discrimination are intercepted so that those reactive oxygen species, which are essential for the signal routes in the cell, are affected. Thus, there is serious doubt that the theory described in EP-A 1 382 327 can really be carried out in practice. After all, the experts are hardly able to identify and select the really useful individual substances from the large number of allegedly active classes of substances.

According to the invention, however, the objective described above is carried out in a new and unexpectedly effective and, in particular, reproducible manner:

Surprisingly, it was found that certain reactive proteins, which are involved in the mt-DNA sections damaged by the repair system described above and thus are active in the mitochondria, develop an excellent protective action for human skin cells through mt-DNA, whereby this action can be further increased considerably by modification. The enzymes of polymerase gamma and telomerase reverse transcriptase have proved effective especially as mitochondrially active proteins.

If these proteins are transported to the mitochondria of the skin cells by suitable topical carriers in the skin, they unfold the requested action against an attack of reactive oxygen species (ROS) in a very specific manner, i.e. those oxygen species, which are required for the function of their signal routes in the cell, remain undamaged. The mechanism of the action is not known yet, although the enzymes mentioned have been examined extensively all over the world, not only within the framework of AIDS research.

The discovery of this new type of action is extraordinarily surprising and the observed effect is completely out of line of the known state of technology.

An especially effective method of preparation of the corresponding compositions is achieved, if the proteins mentioned are packed into liposomes because they permit an especially easy mitochondrial import through the cell membrane. In endothelial cells, an especially good protective effect was found with mitochondrial telomerase reverse transcriptase (mt-TERT).

The object of this invention is a topical composition to be applied to humans consisting of a hypoallergenic carrier and of at least one homogeneously integrated mitochondrial-membrane penetrating protein. These proteins are selected from a number of enzymes which are involved in the natural repair process of mt-DNA. The enzymes of polymerase gamma and mt-TERT are preferred.

Another object of the invention is the use of at least one mitochondrial-membrane penetrating protein from the series of the enzymes involved in the natural repair process of mt-DNA for the protection against and treatment of cell aging of the human skin.

Another object of the invention is the use of at least one mitochondrial-membrane penetrating protein from the series of the enzymes involved in the natural repair process of mt-DNA for the production of a topical agent for the prevention and treatment of cell aging of the human skin.

For the production of the composition according to the invention, the proteins mentioned above are integrated into a suitable carrier and, if and when required, are mixed with the usual auxiliary agents as homogeneously as possible.

Emulsifying agents, solvents, thickeners, filling materials, stabilisers, preservatives, antioxidants and perfumes may by used as auxiliary agents supporting a topical application.

Surface-active agents, such as polyoxyethylene sorbitan acid and ester or salts of bile acid, may be used to potentially improve bio-availability. In addition, suitable active components can be added for the protection of skin. These components include, for example, vitamins, antibacterial, fungistatic or fungicidal agents, provided they are compatible with the mitochondrial proteins used as active components. As already mentioned, the protein correspondingly packed in liposomes are especially effective.

The proteins and/or enzymes used in conformity with the invention are known and commercially available substances.

Another objective of the present invention is to achieve an effective protection of the mitochondria in cells of the human skin by means of the composition topically administered and containing the mitochondrial proteins described.

The objective is achieved by the fact that mitochondrial proteins, which are significant for the human respiratory chain, and which are able not only to repair the human mt-DNA but also to protect it against attacks by noxae, are brought into contact in form of a topical preparation and to have it take effect.

Such a protective mechanism and effective action of mitochondrial-membrane penetrating proteins on mitochondria have not been known up to now.

An application, which has been described above, prepared in conformity with the invention is preferred.

The application of a composition prepared in conformity with the invention, which contains at least one mitochondrial-membrane penetrating protein as active component, such as in the form of a cream, emulsion or lotion on the skin or the epidermis, should be modelled on the respective rationale and is random in this sense as long as the interaction between the active components in conformity with the invention and the mitochondria is ensured and that the active component can act on the mitochondria as intensively as possible through the selected galenics.

The mitochondrial-membrane penetrating proteins administered in conformity with the invention can be processed into all formulations suitable for the application on the human skin. Said formulations are, for example, tinctures, hydrogels, oil in water emulsions, water in oil emulsions, lotions, creams, ointments or sprays. The most favourable concentration is the range of 0.01 to 20.0 weight percent relative to the weight of the support material used. The preferred concentrations are 0.1 to 10 weight percent.

In general, the active components in conformity with the invention can be processed galenically using conventional hypoallergenic and pharmacologically harmless auxiliary and additive substances in well-known manner. Such additive substances are, for example emulsifying agents, solvents, thickeners, filling materials, stabilisers, preservatives, antioxidants or perfumes. Using surface-active agents, such as polyoxyethylene sorbital acid and esters or salts of the bile acid, bio-availability may also be improved, if and when possible.

Apart from the mitochondrial-membrane penetrating proteins, other suitable active ingredients may also be employed for skin protection, which include, for example, vitamins, inorganic or organic ultraviolet filters as well as anti-bacterial, fungistatic or fungicide agents, provided they do not affect the mitochondrial-membrane penetrating proteins in conformity with the invention.

In order to include insoluble matters, such as inorganic oxides or pigments, dispersing agents, such as poly-acrylate, lignin, tannate or other derivates are added, if and when requested. Colloidal silicon oxide, for example, is used as thickening agent. Hydrogels can be produced with hydrophilic organic solvents, such as glycerine, glycol or with aliphatic alcohols. Moreover, in conformity with the invention, it is possible that the active components in conformity with the invention are used in the form of active-component-containing liposomes or microsomes or as liposomal or microsomal encapsulated active components apart from other auxiliary and other active components.

The following examples are used to explain the invention, but shall not limit the said invention in any way whatsoever.

### EXAMPLE 1

### Skin Milk

| Constituent | Content in weight percent |
|---|---|
| Water | 78.8 |
| Mt-TERT | 5.0 |
| Multivalent alcohols | 3.2 |
| Solid thickener | 0.3 |
| Preservatives | 0.2 |
| Antioxidants | 0.1 |
| Perfume oil | 0.6 |
| UV absorber | 5.0 |

### EXAMPLE 2

### Skin Cream

| Constituent | Content in weight percent |
|---|---|
| Water | 56.6 |
| Polymerase Gamma | 8.8 |
| Fat base | 26.0 |
| Multivalent alcohols | 4.0 |
| Solid thickener | 0.5 |
| Preservatives | 0.5 |
| Antioxidants | 0.1 |
| Perfume oil | 0.3 |
| UV absorber | 3.2 |

1) Dermatological or cosmetic preparation for improving the protection of human skin cells against deleterious influences caused by oxidative noxae and ultraviolet irradiation, consisting of an active component and conventional pharmacologically tolerable excipients characterised in that the active component is at least one mitochondrial-membrane penetrating protein.

2) Preparation as set forth in claim 1 characterised in that the active component is mitochondrial telomerase reverse transcriptase.

3) Preparation as set forth in claim 1 characterised in that the active component is polymerase gamma.

4) Application of mitochondrial-membrane penetrating protein for improving the protection of human skin cells against deleterious influences caused by oxidative noxae and ultraviolet irradiation.

5) Application as set forth in claim 4 characterised in that the mitochondrial-membrane penetrating protein is mitochondrial telomerase reverse transcriptase.

6) Application as set forth in claim 4 characterised in that the mitochondrial protein is polymerase gamma.

7) Cosmetic process for improving the protection of the human skin cells again deleterious influences caused by oxidative noxae and ultraviolet irradiation by putting on an active component with conventional pharmacologically tolerable excipients characterised in that the active component is at least one mitochondrial-membrane penetrating protein.

8) Method as set forth in claim 7 characterised in that the mitochondrial-membrane penetrating protein is a mitochondrial telomerase reverse transcriptase.

9) Method as set forth in claim 7 characterised in that the mitochondrial-membrane penetrating protein is polymerase gamma.
